(19) United States
(12) Patent Application Publication
(10) Pub. No.: US 2025/0049471 A1
(43) Pub. Date: Feb. 13, 2025

(54) PUNCTURE DEVICE

(71) Applicant: Asahi Intecc Co., Ltd., Seto-shi (JP)

(72) Inventors: Takeru FUKUNAGA, Seto-shi (JP); Yukihiro FUSEYA, Seto-shi (JP); Tomoki KOSUGI, Seto-shi (JP); Yuki TAKEBE, Seto-shi (JP)

(73) Assignee: Asahi Intecc Co., Ltd., Seto-shi (JP)

(21) Appl. No.: 18/885,827

(22) Filed: Sep. 16, 2024

Related U.S. Application Data
(63) Continuation of application No. PCT/JP2023/002360, filed on Jan. 26, 2023.

(30) Foreign Application Priority Data
Apr. 12, 2022 (JP) 2022-065916

(51) Int. Cl. A61B 17/34

(52) U.S. Cl. CPC A61B 17/3478

(57) ABSTRACT

A puncture device includes a puncture portion having a needle tip with a sharp distal end, and a shaft portion on a proximal end side of the puncture portion. The shaft portion includes a core member and an outer layer body arranged on an outer peripheral surface of the core member.

### CROSS-REFERENCE TO RELATED APPLICATIONS

This application is a continuation application of International Application No. PCT/JP2023/002360, filed Jan. 26, 2023, which claims priority to Japanese Patent Application No. 2022-065916, filed Apr. 12, 2022. The contents of these applications are incorporated herein by reference in their entirety.

### TECHNICAL FIELD

The present disclosure relates to a puncture device for an endoscope.

### BACKGROUND ART

Procedures called interventional EUS in which the inside of an abdominal cavity is observed with an ultrasonic endoscope (EUS) via a digestive tract such as a stomach or an intestine and a disease in the abdominal cavity is treated are conventionally known. As one of such procedures, there is endoscopic ultrasound-guided biliary drainage (EUS-BD) in which a puncture of a bile duct is performed from the inside of a stomach or a duodenum and a stent is indwelled in the bile duct in which a flow of bile is deteriorated. For example, in the case of treatment of performing a puncture of a bile duct from the inside of a stomach, which is called EUS-HGS, a puncture device for an endoscope, which includes a sheath and a puncture needle, is used together with an ultrasonic endoscope. In the EUS-HGS, the distal end portion of the puncture needle is pushed out from the distal end of the sheath protruding from the distal end of the ultrasonic endoscope, a puncture of a stomach and a liver is performed from the inside of the stomach to an intrahepatic bile duct, and a fistula is formed from the stomach to the bile duct. For example, Patent Literature 1 discloses a puncture device for an endoscope, which is used in such treatment.

### CITATION LIST

### Patent Literature

Patent Literature 1: JP 2010-194011 A

### SUMMARY

### Technical Problems

In the puncture device for an endoscope of Patent Literature 1, a puncture needle using a thin stainless steel pipe or another metal pipe material is inserted and arranged in a flexible sheath to be inserted into the inside (treatment tool insertion channel) of an ultrasonic endoscope. In performing puncture treatment using such puncture device for an endoscope, in order to stabilize the distal end of the endoscope, a puncture target site is sometimes punctured in a state where the endoscope is sufficiently up-angled (that is, a state where the distal end portion of the endoscope is bent). However, when the puncture needle is pushed out in a state where the endoscope is angled, the puncture needle itself may have a tendency to bend and be deformed. When the puncture needle is bent, the straight advancing stability is lost, and as a result, the puncture needle becomes difficult to aim at the puncture target site.

The present disclosure has been made in view of such a point, and embodiments are directed to providing a puncture device having high puncture performance and safety.

### Solutions to Problems

In order to achieve the above, the present disclosure provides a puncture device including: a puncture portion having a needle tip with a sharp distal end; and a shaft portion provided on a proximal end side of the puncture portion, in which the shaft portion includes a core member and an outer layer body arranged on an outer peripheral surface of the core member.

Accordingly, in the configuration in which the outer layer body is arranged on the outer peripheral surface of the core member, the flexibility of the shaft portion can be easily adjusted by changing the diameter of the core member and the thickness of the outer layer body. Therefore, the shaft portion can be formed to be more flexible than the puncture portion, and as a result, the puncture device having high puncture performance and safety can be provided.

In addition, in the configuration in which the outer layer body is arranged on the outer peripheral surface of the core member, the outer diameter of the puncture portion and the outer diameter of the shaft portion can be configured to be uniform by changing the diameter of the core member and the thickness of the outer layer body. As a result, in a state where the puncture portion and the shaft portion are arranged in an inner sheath, an unnecessary clearance (gap) is eliminated between the outer periphery of the puncture portion and the shaft portion and the inner peripheral surface of the inner sheath. Therefore, when a puncture is performed using the puncture device, unnecessary motions such as flexure of the shaft portion in the inner sheath can be prevented.

Accordingly, a puncture device having both high puncture performance and safety can be provided.

### BRIEF DESCRIPTION OF DRAWINGS

FIG. 1 is an explanatory view illustrating an entire structure of a puncture device according to a first embodiment.

FIG. 2 is an explanatory view illustrating a structure of a distal end portion of the puncture device according to the first embodiment.

FIG. 3 is an explanatory view illustrating a structure of a distal end portion of a puncture device according to a second embodiment.

FIG. 4 is an explanatory view illustrating a structure of a distal end portion of a puncture device according to a third embodiment.

### DETAILED DESCRIPTION

Hereinafter, embodiments of the present disclosure will be described with reference to the drawings. The present disclosure is not limited only to the embodiments described below, and the described embodiments are merely examples for describing the technical features of the present disclosure. The shapes and dimensions illustrated in the drawings are merely illustrated to facilitate understanding of the contents of the present disclosure, and do not accurately reflect actual shapes and dimensions.

### First Embodiment

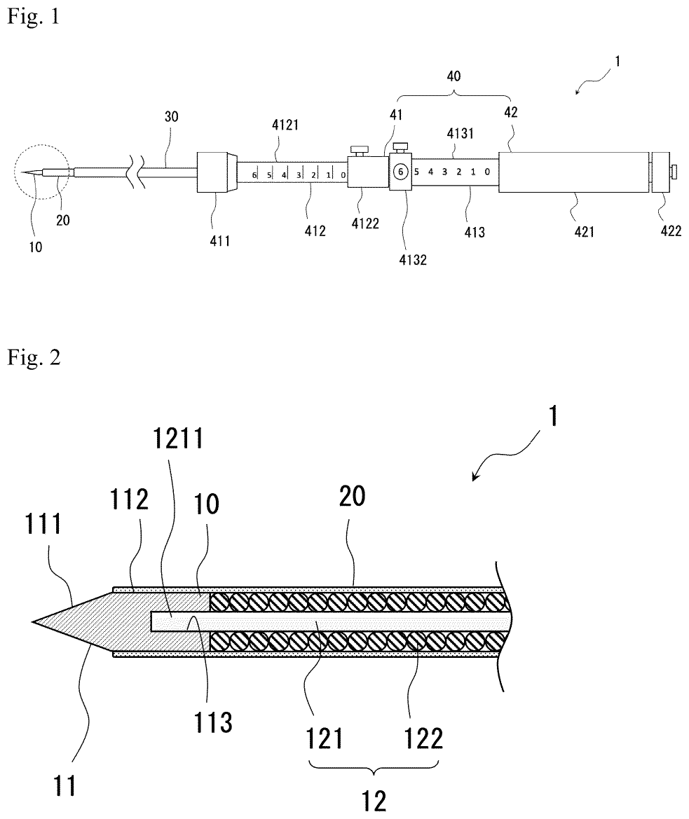

FIG. 1 is an explanatory view illustrating an entire structure of a puncture device 1 according to a first embodiment, and FIG. 2 is an explanatory view illustrating a structure of a distal end portion (a portion surrounded by a dashed line in FIG. 1) of the puncture device 1.

In the present description, the “distal end side” means a direction along the axial direction of the puncture device 1 and a direction in which the puncture device 1 and an endoscope (not illustrated) on which the puncture device 1 is mounted proceed toward a puncture target site. The “proximal end side” means a direction along the axial direction of the puncture device 1 and a direction opposite to the above-described distal end side. The “distal end” refers to, in any given member or site, an end portion on the distal end side, and the “proximal end” refers to, in any given member or site, an end portion on the proximal end side. The “distal end portion” refers to, in any given member or site, a site including the distal end thereof and extending from the distal end toward the proximal end side to the middle of the member or the like, and the “proximal end portion” refers to, in any given member or site, a site including the proximal end thereof and extending from the proximal end toward the distal end side to the middle of the member or the like. In FIG. 1, the left side in the drawing is the “distal end side” to be inserted into a body, and the right side in the drawing is the “proximal end side” to be operated by a professional.

As illustrated in FIG. 1, the puncture device 1 includes a needle member 10, an inner sheath 20, an outer sheath 30, and an operation part 40. The puncture device 1 is used when, for example, in the procedure of EUS-BD, the distal end of the needle member 10 is caused to penetrate the peripheral walls of luminal organs (for example, a stomach or a liver) to be bypass-connected to form penetration-pores, and a guide wire is inserted between the luminal organs via the penetration-pores. By inserting the outer sheath 30 (and the inner sheath 20 and the needle member 10 incorporated in the inner cavity of the outer sheath 30) into a treatment tool insertion channel of an endoscope (not illustrated) and operating the operation part 40, the puncture device 1 is configured such that the outer sheath 30 can slide (relatively move) in the axial direction with respect to the treatment tool insertion channel of the endoscope. The distal end portion of the puncture device 1 protrudes from the distal end of the treatment tool insertion channel of the endoscope.

The inner sheath 20 is an elongated tubular member having flexibility, and the needle member 10 is inserted in the inner cavity thereof. The inner sheath 20 is formed to have an inner diameter equal to or slightly larger than the outer diameter of the needle member 10 such that the needle member 10 can slide (relatively move) in the axial direction in the inner cavity of the inner sheath 20. The inner sheath 20 is formed to have an outer diameter slightly smaller than the inner diameter of the outer sheath 30 such that the inner sheath 20 can slide (relatively move) in the axial direction in the inner cavity of the outer sheath 30 to be described below.

The material forming the inner sheath 20 is not particularly limited as long as the material has a strength capable of withstanding the sliding of the needle member 10 in the inner cavity of the inner sheath 20 and has biocompatibility, and for example, various resin materials such as polyethylene, polypropylene, polyvinyl chloride, polystyrene, acrylic resin, phenol resin, melamine resin, polyimide, polyamide, polycarbonate, polyether sulfone, polyether ether ketone, and polytetrafluoroethylene, and various thermoplastic elastomers such as polystyrene, polyolefin, polyurethane, polyester, polyamide, and polybutadiene can be used. Two or more of these materials may be used in combination.

By operating the operation part 40, the inner sheath 20 can slide (relatively move) in the axial direction with respect to the outer sheath 30.

The outer sheath 30 is an elongated tubular member having flexibility, and the inner sheath 20 is inserted in the inner cavity thereof. The outer sheath 30 is formed to have an inner diameter slightly larger than the outer diameter of the inner sheath 20 such that the inner sheath 20 can slide (relatively move) in the axial direction in the inner cavity of the outer sheath 30. The outer diameter of the outer sheath 30 has a size that can be inserted into the treatment tool insertion channel of the endoscope (not illustrated).

The material forming the outer sheath 30 is not particularly limited as long as the material has a strength capable of withstanding the sliding of the inner sheath 20 in the inner cavity of the outer sheath 30 and has biocompatibility, and for example, the materials exemplified as the material forming the inner sheath 20 can be used.

By operating the operation part 40, the outer sheath 30 can slide (relatively move) in the axial direction with respect to the treatment tool insertion channel of the endoscope.

The operation part 40 is configured by a plurality of members formed of, for example, a resin material, and includes an operation part main body 41 to which the proximal end portion of the outer sheath 30 is fixed, and a slider part 42 provided to be slidable with respect to the operation part main body 41. All of the members configuring the operation part 40 may be formed of the same material, or some of the members may be formed of a different material.

The operation part main body 41 has an elongated hollow cylindrical shape as a whole, and includes a connection portion 411 connected and fixed to an inlet of the treatment tool insertion channel of the endoscope, an outer sheath length adjustment portion 412 connected to the proximal end side of the connection portion 411, and an inner sheath length adjustment portion 413 connected to the proximal end side of the outer sheath length adjustment portion 412.

The connection portion 411 is fixed to the distal end portion of a scale portion 4121 of the outer sheath length adjustment portion 412 by a means of adhesion or the like, and the proximal end portion of the outer sheath 30 is fixed thereto. The connection portion 411 is connected and fixed to the inlet of the treatment tool insertion channel of the endoscope, so that the puncture device 1 can be fixed to the endoscope.

The outer sheath length adjustment portion 412 is a mechanism for adjusting the positional relationship between the endoscope and the outer sheath 30, and has the scale portion 4121 and a stopper 4122. The stopper 4122 is arranged to be slidable with respect to the scale portion 4121. The scale portion 4121 has a plurality of scales serving as indexes for setting the arrangement position of the stopper 4122, and the scales are arranged at predetermined intervals on the outer peripheral surface of the scale portion 4121. Each scale represents a length from the distal end of the connection portion 411 to the distal end of the outer sheath 30.

The inner sheath length adjustment portion 413 is a mechanism for adjusting the positional relationship between the outer sheath 30 and the inner sheath 20 (and the needle member 10 incorporated in the inner cavity of the inner sheath 20), and has a scale portion 4131 and a stopper 4132. The stopper 4132 is arranged to be slidable with respect to the scale portion 4131. The scale portion 4131 has a plurality of scales serving as indexes for setting the arrangement position of the stopper 4132, and the scales are arranged at predetermined intervals on the outer peripheral surface of the scale portion 4131. Each scale represents a protrusion length of the inner sheath 20 (and the needle member 10 incorporated in the inner cavity of the inner sheath 20) from the distal end of the outer sheath 30. For example, the stopper 4132 is arranged at a position of the scale corresponding to the numerical value “1,” and is fixed by turning a screw. In this state, when the slider part 42 is slid to the distal end side, the distal end of the inner sheath 20 (and the needle member 10) can be protruded from the distal end of the outer sheath 30 by the length (1 cm) represented by the scale.

The slider part 42 has an elongated hollow cylindrical shape as a whole, and includes a handle part 421 to be grasped by a user, and a needle fixation portion 422 which is connected to the proximal end side of the handle part 421 and to which the proximal end portion of the needle member 10 is fixed.

As illustrated in FIG. 2, the needle member 10 according to the present embodiment includes a puncture portion 11 having a solid structure, which has a needle tip 111 with a sharp distal end, and a shaft portion 12 provided on the proximal end side of the puncture portion 11. The length of the puncture portion 11 is about from 5 to 80 mm, and the length of the shaft portion 12 varies depending on the use or the like of the puncture device 1.

The puncture portion 11 includes the needle tip 111 having a substantially triangular pyramid shape with a triangular pointed distal end, and a columnar base portion 112 formed integrally with the needle tip 111, and an insertion hole 113 opened in a direction toward the shaft portion 12 (proximal end direction) is formed on the proximal end side of the base portion 112.

The material forming the puncture portion 11 is not particularly limited as long as the material has a hardness and strength to the extent that the needle tip 111 can penetrate the puncture target site to form a penetration-pore and has biocompatibility, and for example, metal materials such as stainless steel, titanium, a nickel-titanium alloy, and a cobalt-chromium alloy can be used.

The maximum outer diameter of the needle tip 111 of the puncture portion 11 needs to be equal to or smaller than the outer diameter of the base portion 112 such that the puncture portion 11 is not caught by the inner sheath 20 when the needle member 10 is pulled out from the inner sheath 20.

The shaft portion 12 has a substantially uniform diameter over the entire length, and includes a core member 121 having a substantially perfect circular cross-section, and an outer layer body 122 arranged on the outer peripheral surface of the core member 121. The outer diameter of the outer layer body 122 is substantially the same as the outer diameter of the base portion 112 of the puncture portion 11, and the needle member 10 is inserted in the inner cavity of the inner sheath 20 such that the outer peripheral surface from the base portion 112 of the puncture portion 11 to the entire outer layer body 122 is in contact with the inner peripheral surface of the inner sheath 20.

A distal end portion 1211 of the core member 121 is inserted in the insertion hole 113 formed in the base portion 112 of the puncture portion 11. Accordingly, at least a part of the core member 121 (the distal end portion 1211) is positioned inside the puncture portion 11. The distal end portion 1211 of the core member 121 may be fixed to the base portion 112 of the puncture portion 11 using a known fixing means. The material forming the core member 121 is not particularly limited, and for example, the metal materials same as the material forming the puncture portion 11 can be used. The core member 121 may be entirely formed of the same material, or may be partially formed of a different material. As described above, by configuring the needle member 10 such that at least a part of the core member 121 (the distal end portion 1211) is positioned inside the puncture portion 11, the puncture portion 11 and the shaft portion 12 can be joined more stably than when the puncture portion 11 and the shaft portion 12 are simply brought into contact with each other and joined at a surface.

The outer layer body 122 of the shaft portion 12 is a wire rope which is obtained by helically twisting a plurality of wires (strand or strand group) and is arranged around the core member 121. The material forming the outer layer body 122 is not particularly limited, and for example, the metal materials same as the material forming the puncture portion 11 can be used. The outer layer body 122 may be entirely formed of the same material, or may be partially formed of a different material. By adopting a wire rope as the outer layer body 122, the outer surface of the shaft portion 12 can be provided with irregularities. As a result, the contact area with the inner peripheral surface of the inner sheath 20 is reduced, and the slidability of the needle member 10 is improved.

The distal end of the outer layer body 122 (the distal end surface of the outer layer body 122) is in contact with the proximal end of the puncture portion 11 (the proximal end surface of the base portion 112 of the puncture portion 11), and the proximal end of the puncture portion 11 and the distal end of the outer layer body 122 are fixed to each other using a known joining means such as an adhesive, brazing, pressure welding, or YAG laser welding.

In the configuration in which the outer layer body 122 is arranged on the outer peripheral surface of the core member 121 as in the shaft portion 12 of the needle member 10 described above, the flexibility of the shaft portion 12 can be easily adjusted by changing the diameter of the core member 121 and the thickness of the outer layer body 122. Therefore, the shaft portion 12 can be formed to be more flexible than the puncture portion 11, and as a result, the puncture device 1 having high puncture performance and safety can be provided.

In addition, in the configuration in which the outer layer body 122 is arranged on the outer peripheral surface of the core member 121, the outer diameter of the puncture portion 11 and the outer diameter of the shaft portion 12 can be configured to be uniform (that is, the outer diameter of the needle member 10 can be configured to be uniform as a whole) by changing the diameter of the core member 121 and the thickness of the outer layer body 122. As a result, in a state where the needle member 10 (the puncture portion 11 and the shaft portion 12) is arranged in the inner sheath 20, an unnecessary clearance (gap) is eliminated between the outer periphery of the needle member 10 (the puncture portion 11 and the shaft portion 12) and the inner peripheral surface of the inner sheath 20. Therefore, when a puncture is performed using the puncture device 1, unnecessary motions such as flexure of the shaft portion 12 in the inner sheath 20 can be prevented.

When the shaft portion 12 is formed to be more flexible than the puncture portion 11, even if the needle member 10 is pushed out in a state where the endoscope is up-angled in performing puncture treatment using the puncture device 1, the needle member 10 itself can be prevented from being bent and deformed. Therefore, a penetration-pore can be completely and smoothly formed in the puncture target site, and the puncture device 1 having high puncture performance and safety can be provided.

### Second Embodiment

In a puncture device 1A according to a second embodiment, the needle member 10 of the puncture device 1 according to the first embodiment is replaced with a needle member 10A having a structure described below. Since the structures of the inner sheath 20, the outer sheath 30, and the operation part 40 are the same as those of the puncture device 1 according to the first embodiment, the description thereof will be omitted.

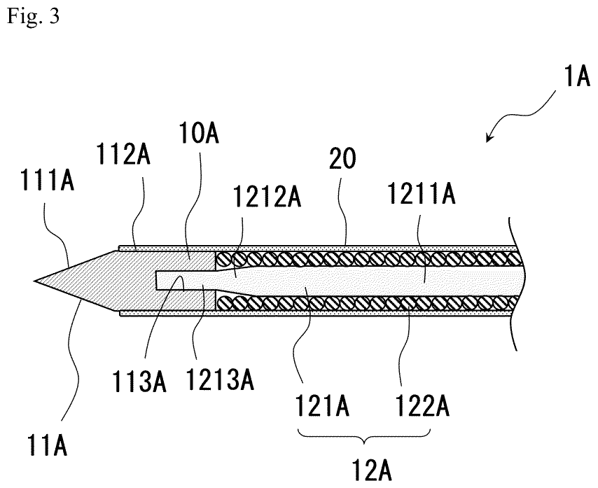

FIG. 3 is an explanatory view illustrating a structure of a distal end portion of the puncture device 1A according to the second embodiment. As illustrated in FIG. 3, the needle member 10A according to the present embodiment includes a puncture portion 11A having a solid structure, which has a needle tip 111A with a sharp distal end, and a shaft portion 12A provided on the proximal end side of the puncture portion 11A. The length of the puncture portion 11A is about from 5 to 80 mm, and the length of the shaft portion 12A varies depending on the use or the like of the puncture device 1A.

The puncture portion 11A includes the needle tip 111A having a substantially triangular pyramid shape with a triangular pointed distal end, and a columnar base portion 112A formed integrally with the needle tip 111A, and an insertion hole 113A opened in a direction toward the shaft portion 12A (proximal end direction) is formed on the proximal end side of the base portion 112A.

The material forming the puncture portion 11A is not particularly limited as long as the material has a hardness and strength to the extent that the needle tip 111A can penetrate the puncture target site to form a penetration-pore and has biocompatibility, and for example, metal materials such as stainless steel, titanium, a nickel-titanium alloy, and a cobalt-chromium alloy can be used.

The shaft portion 12A includes a core member 121A having a substantially perfect circular cross-section, in which a core distal end portion 1213A has a diameter smaller than that of a core main body part 1211A, and an outer layer body 122A arranged on the outer peripheral surface of the core member 121A. The outer diameter of the outer layer body 122A is substantially the same as the outer diameter of the puncture portion 11A, and the needle member 10A is inserted in the inner cavity of the inner sheath 20 such that the outer peripheral surface from the puncture portion 11A to the entire outer layer body 122A is in contact with the inner peripheral surface of the inner sheath 20.

The core member 121A includes the substantially columnar core main body part 1211A, a core tapered portion 1212A having a tapered shape (truncated cone shape) in which the outer diameter gradually decreases, and the substantially columnar core distal end portion 1213A having an outer diameter smaller than that of the core main body part 1211A. The outer diameter of the core main body part 1211A is formed to be equal to the inner diameter of the outer layer body 122A, and the outer diameter of the core distal end portion 1213A is formed to be equal to the inner diameter of the insertion hole 113A of the puncture portion 11A.

The distal end portion 1213A of the core member 121A is inserted in the insertion hole 113A of the puncture portion 11A. Accordingly, at least a part of the core member 121A (the distal end portion 1213A) is positioned inside the puncture portion 11A. The distal end portion 1213A of the core member 121A may be fixed to the puncture portion 11A using a known fixing means. The material forming the core member 121A is not particularly limited, and for example, the metal materials same as the material forming the puncture portion 11A can be used. The core member 121A may be entirely formed of the same material, or may be partially formed of a different material.

The outer layer body 122A of the shaft portion 12A is a wire rope which is obtained by helically twisting a plurality of wires (strand or strand group) and is arranged around the core member 121A. The material forming the outer layer body 122A is not particularly limited, and for example, the metal materials same as the material forming the puncture portion 11A can be used. The outer layer body 122A may be entirely formed of the same material, or may be partially formed of a different material.

The distal end of the outer layer body 122A is in contact with the proximal end of the puncture portion 11A, and the proximal end of the puncture portion 11A and the distal end of the outer layer body 122A are fixed to each other using a known joining means such as an adhesive, brazing, pressure welding, or YAG laser welding.

As in the shaft portion 12A according to the present embodiment, by making only the diameter of the distal end portion of the core member smaller than the overall diameter and forming the outer layer body to be thin, the core member having a large diameter can be adopted for the shaft portion while maintaining the outer diameter of the overall shaft portion. Therefore, the degree of freedom of adjusting the flexibility imparted to the shaft portion can be increased.

### Third Embodiment

In a puncture device 1B according to a third embodiment, the needle member 10 of the puncture device 1 according to the first embodiment is replaced with a needle member 10B having a structure described below. Since the structures of the inner sheath 20, the outer sheath 30, and the operation part 40 are the same as those of the puncture device 1 according to the first embodiment, the description thereof will be omitted.

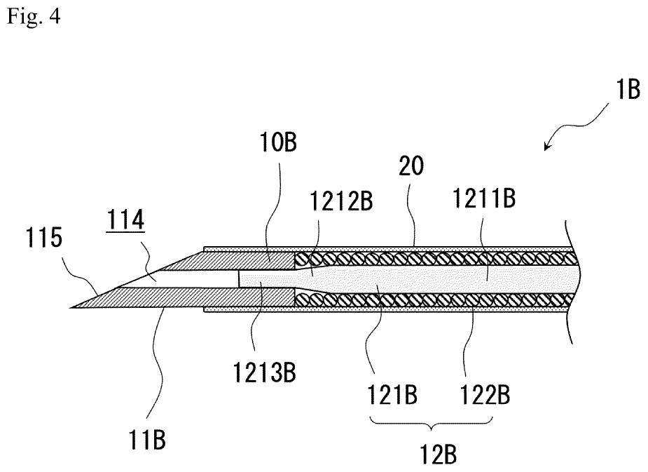

FIG. 4 is an explanatory view illustrating a structure of a distal end portion of the puncture device 1B according to the third embodiment. As illustrated in FIG. 4, the needle member 10B according to the present embodiment includes a puncture portion 11B having a hollow structure, and a shaft portion 12B provided on the proximal end side of the puncture portion 11B. The length of the puncture portion 11B is about from 5 to 80 mm, and the length of the shaft portion 12B varies depending on the use or the like of the puncture device 1B.

The puncture portion 11B may have a shape in which a blade surface 115 is formed by cutting the distal end side of a substantially hollow cylindrical member with a through hole 114 having a perfect circular shape in a cross-sectional view along an axis line obliquely with respect to the axis line. Alternatively, the puncture portion 11B may be any type of blade, e.g., scissors, razors, knives, and the like.

The material forming the puncture portion 11B is not particularly limited as long as the material has a hardness and strength to the extent that the blade surface 115 can penetrate the puncture target site to form a penetration-pore and has biocompatibility, and for example, metal materials such as stainless steel, titanium, a nickel-titanium alloy, and a cobalt-chromium alloy can be used.

The shaft portion 12B includes a core member 121B having a substantially perfect circular cross-section, in which a core distal end portion 1213B has a diameter smaller than that of a core main body part 1211B, and an outer layer body 122B arranged on the outer peripheral surface of the core member 121B. The outer diameter of the outer layer body 122B is substantially the same as the outer diameter of the puncture portion 11B, and the needle member 10B is inserted in the inner cavity of the inner sheath 20 such that the outer peripheral surface from the puncture portion 11B to the entire outer layer body 122B is in contact with the inner peripheral surface of the inner sheath 20.

The core member 121B includes the substantially columnar core main body part 1211B, a core tapered portion 1212B having a tapered shape (truncated cone shape) in which the outer diameter gradually decreases, and the substantially columnar core distal end portion 1213B having an outer diameter smaller than that of the core main body part 1211B. The outer diameter of the core main body part 1211B is formed to be equal to the inner diameter of the outer layer body 122B, and the outer diameter of the core distal end portion 1213B is formed to be equal to the inner diameter of the through hole 114 of the puncture portion 11B.

The distal end portion 1213B of the core member 121B is inserted in the through hole 114 of the puncture portion 11B. Accordingly, at least a part of the core member 121B (the distal end portion 1213B) is positioned inside the puncture portion 11B. The distal end portion 1213B of the core member 121B may be fixed to the puncture portion 11B using a known fixing means. The material forming the core member 121B is not particularly limited, and for example, the metal materials same as the material forming the puncture portion 11B can be used. The core member 121B may be entirely formed of the same material, or may be partially formed of a different material.

The outer layer body 122B of the shaft portion 12B is a wire rope which is obtained by helically twisting a plurality of wires (strand or strand group) and is arranged around the core member 121B. The material forming the outer layer body 122B is not particularly limited, and for example, the metal materials same as the material forming the puncture portion 11B can be used. The outer layer body 122B may be entirely formed of the same material, or may be partially formed of a different material.

The distal end of the outer layer body 122B is in contact with the proximal end of the puncture portion 11B, and the proximal end of the puncture portion 11B and the distal end of the outer layer body 122B are fixed to each other using a known joining means such as an adhesive, brazing, pressure welding, or YAG laser welding.

As in the shaft portion 12B according to the present embodiment, by making only the diameter of the distal end portion of the core member smaller than the overall diameter and forming the outer layer body to be thin, the core member having a large diameter can be adopted for the shaft portion while maintaining the outer diameter of the overall shaft portion. Therefore, the degree of freedom of adjusting the flexibility imparted to the shaft portion can be increased.

In addition, as in the puncture portion 11B according to the present embodiment, the puncture portion has a hollow structure in which the through hole is formed along the axis line, so that the puncture portion can be provided with a function as a biopsy needle that takes a piece of tissue of the puncture target site.

Although the puncture device according to the present disclosure has been described with reference to the drawings, the present disclosure is not limited to the above-described embodiments, and various modifications can be made. For example, although a wire rope formed by helically twisting a plurality of wires is adopted for the outer layer body 122 of the shaft portion 12 in the above-described embodiment, the present disclosure is not limited thereto, and a tube body or a coil body formed of, for example, a metal material or a resin material may be adopted for the outer layer body 122 in consideration of the material adopted for the core member 121 of the shaft portion 12 such that the flexibility of the shaft portion 12 is higher than that of the puncture portion 11.

In addition, regarding the joining of the puncture portion 11 and the shaft portion 12, the joint strength may be increased by laser spot welding the distal end portion 1211 of the core member 121 inserted in the insertion hole 113 of the puncture portion 11 from the outer surface of the puncture portion 11, or a groove may be cut in the puncture portion 11 from the outer surface and the distal end portion 1211 of the core member 121 may be inserted and embedded in the groove.

In the above-described embodiment, a distal end portion of the core member may be positioned inside the puncture portion.

Accordingly, the puncture portion and the shaft portion can be joined more stably than when the puncture portion and the shaft portion are simply brought into contact with each other and joined at a surface.

In the above-described embodiment, the distal end portion of the core member may be inserted in an insertion hole formed in the puncture portion.

In the above-described embodiment, a proximal end of the puncture portion may be in contact with a distal end of the outer layer body 4).

In the above-described embodiment, an outer diameter of the puncture portion may be substantially the same as an outer diameter of the shaft portion.

### DESCRIPTION OF REFERENCE NUMERALS

1, 1A, 1B puncture device
10, 10A, 10B needle member
11, 11A, 11B puncture portion
111, 111A needle tip
112, 112A base portion
113, 113A insertion hole
114 through hole
115 blade surface
12, 12A, 12B shaft portion
121, 121A, 121B core member
122, 122A, 122B outer layer body
20 inner sheath
30 outer sheath
40 operation part

1. A puncture device comprising:
a puncture portion having a sharp distal end; and
a shaft portion provided on a proximal end side of the puncture portion, wherein
the shaft portion includes a core member and an outer layer body on an outer peripheral surface of the core member.

2. The puncture device according to claim 1, wherein
a distal end portion of the core member is positioned inside the puncture portion.

3. The puncture device according to claim 2, wherein
the distal end portion of the core member is inserted in an insertion hole in the puncture portion.

4. The puncture device according to claim 1, wherein
a proximal end of the puncture portion is in contact with a distal end of the outer layer body.

5. The puncture device according to claim 1, wherein
an outer diameter of the puncture portion is substantially the same as an outer diameter of the shaft portion.

6. The puncture device according to claim 1, wherein the sharp distal end of the puncture portion is a needle tip.

7. The puncture device according to claim 1, wherein the sharp distal end of the puncture portion is a blade.

8. The puncture device according to claim 1, wherein flexibility of the shaft portion is higher than that of the puncture portion.

9. The puncture device according to claim 1, wherein the core member includes a core main body and a core distal end portion positioned inside the puncture portion, wherein, a diameter of the core distal end portion is smaller than a diameter of the core main body.

10. The puncture device according to claim 9, wherein the core member further includes a tapered portion between the core main body and the core distal end portion, the tapered portion having a diameter decreasing from the diameter of the core main body to the diameter of the core distal end portion.

11. The puncture device according to claim 1, wherein a distal end portion of the core member extends into the puncture portion beyond the outer layer body.

12. The puncture device according to claim 1, further comprising an inner sheath that houses the puncture portion and the shaft portion, wherein a distal end of puncture portion protrudes beyond the inner sheath.

13. The puncture device according to claim 12, wherein the inner sheath completely surrounds the shaft portion.

14. The puncture device according to claim 12, wherein an outer surface of the outer layer body in contact with the inner sheath has an irregular surface.

15. The puncture device according to claim 12, further comprising an outer sheath having a larger inner diameter than an outer diameter of the inner sheath to allow the inner sheath to relatively move in an axial direction, wherein a distal end of the inner sheath protrudes from a distal end of the outer sheath.

16. The puncture device according to claim 15, wherein the outer sheath has a diameter to be coupled with an insertion channel of a treatment tool.

17. The puncture device according to claim 16, further comprising an operation portion that includes a connection portion to be connected to the insertion channel of the treatment tool, the operation portion to move outer sheath relative to the insertion channel of the treatment tool in the axial direction.

18. The puncture device according to claim 17, wherein the operation portion includes a scale that represents a length from a distal end of the connection portion to the distal end of the outer sheath.
